(19) United States
(12) Patent Application Publication
(10) Pub. No.: US 2016/0000979 A1
(43) Pub. Date: Jan. 7, 2016

(54) BIOACTIVE IMPLANT AND MANUFACTURING METHOD OF BIOACTIVE IMPLANT

(71) Applicant: MITSUBISHI PAPER MILLS LIMITED, Sumida-ku, Tokyo (JP)

(72) Inventor: Akira FURUKAWA, Sumida-ku, Tokyo (JP)

(73) Assignee: MITSUBISHI PAPER MILLS LIMITED, Sumida-ku, Tokyo, OT (JP)

(21) Appl. No.: 14/770,215

(22) PCT Filed: Feb. 14, 2014
(86) PCT No.: PCT/JP2014/053525

§ 371 Date: Aug. 25, 2015

(30) Foreign Application Priority Data
Feb. 26, 2013 (JP) 2013-035236
Jan. 16, 2014 (JP) 2014-005682

(51) Int. Cl. A61L 31/16; A61L 31/10

(52) U.S. Cl. CPC A61L 31/16; A61L 31/10; A61L 2420/02; A61L 2420/06

(57) ABSTRACT

There is provided a bioactive implant that has good bioactivity and can firmly maintain adhesion between a metal substrate or a plastic substrate and an apatite layer and abrasion resistance for an extended period of time. The bioactive implant has the apatite layer containing at least crystalline calcium apatite fine particles, an aqueous urethane resin, and a self-emulsifiable isocyanate compound, on the surface of the metal substrate or the plastic substrate.

### TECHNICAL FIELD

The present invention relates to a bioactive implant which can be used as various living body-embedded type implants, and a manufacturing method of the bioactive implant.

### BACKGROUND ART

Hydroxyapatite, which is one of calcium apatites, is one of calcium phosphate salts constituting bones, teeth and the like. Hydroxyapatite exhibits high biocompatibility, and is thus known as an extremely safe material. Especially, a material on a surface of which hydroxyapatite is applied exhibits good osteoconductivity. For this reason, hydroxyapatite is used for surface treatment of various living body-embedded type implants in which connection with bones is considered as important in various medical apparatuses. This takes advantage of a phenomenon that coating surfaces of various implants with hydroxyapatite leads to extremely good connection between the implants and bones. That is, when hydroxyapatite is applied on surfaces of various implants, various actions proceed in the body so that hydroxyapatite (bioapatite) produced by the living body is precipitated on surfaces of these hydroxyapatite layers. As a result, the implant surfaces and bioapatite are firmly connected with each other via the applied hydroxyapatite layers. This enables extremely good connection between implants and bones.

An example of various living body-embedded type implants, in which connection with bones is considered as important, may include a bioactive implant used in sites to which strong stress is applied, such as joints, tendons, ligaments, spines, and tooth roots. As a substrate for forming such a bioactive implant, a living body-derived material is used. Other examples of the substrate include metal substrates such as titanium and plastic substrates such as polyester, polycarbonate and PEEK (polyether ether ketone). An example of a method for increasing bioactivity by coating a metal substrate surface with hydroxyapatite may include plasma spraying disclosed in PATENT LITERATURE 1, PATENT LITERATURE 2 and the like. In this case, hydroxyapatite is sprayed at high temperature. Consequently, there has been a problem that thermal decomposition is partially initiated. In addition, a hydroxyapatite coat formed by spraying is amorphous and porous. Consequently, there has been a problem that the hydroxyapatite coat gradually dissolves in the living body and falls out from the implant surface. Furthermore, when plasma spraying is similarly attempted to be employed for the purpose of manufacturing a bioactive implant with a plastic substrate, the substrate is required to have high thermal resistance. Consequently, there has been a problem that plasma spraying cannot be employed for the plastic substrate.

An example of a method used for coating the metal substrate or the plastic substrate with calcium apatite includes a method of immersing the substrate in a pseudo body fluid as disclosed in PATENT LITERATURE 3. Another example of the method, particularly for the plastic substrate, includes a method of similarly immersing the substrate in a pseudo body fluid after forming a glass body on a surface constituted by a specific organic polymer compound as disclosed in PATENT LITERATURE 4. An alternative example of the method includes a method of previously forming a calcium phosphate trapping layer by performing an alternate immersion process in which a substrate surface is alternately immersed in an aqueous calcium salt solution and in an aqueous phosphate solution, and subsequently forming an apatite layer on this calcium phosphate trapping layer with an aqueous supersaturated calcium phosphate solution as disclosed in PATENT LITERATURE 5.

The apatite layer is formed on the surface of the metal substrate or the plastic substrate by various known processes as described above. However, when these processes are used for the bioactive implant, serious problems have been raised in some cases. One of the problems is that apatite formed on the surface has low crystallinity. Consequently, there is a problem that calcium apatite is likely to be eluted in the body fluid. Particularly, when the body fluid near the implant is inclined to the acidic side, such as when inflammation is caused around the implant, solubility of calcium apatite increases. This has sometimes caused the apatite layer to be gradually dissolved, and eventually disappear. In such a case, connection between an implant and a bone is lost. As a result, the implant is not fixed to the embedded site. In addition, adhesion at an interface between the apatite layer formed by the above-described process and the metal substrate or the plastic substrate is not sufficient. Accordingly, when stress was repeatedly applied to the implant, the applied apatite layer was sometimes peeled off from the surface of the metal substrate or the plastic substrate. In such a case, even when the bioapatite layer has been formed on the surface of the applied apatite layer in the living body, connection between the implant and the bone is finally lost. Furthermore, the thickness of the apatite layer is difficult to control in the above-described various processes. Therefore, an apatite layer having the most suitable thickness has been difficult to provide on the surface of the metal substrate or the plastic substrate for any purpose.

PATENT LITERATURE 6 discloses a method of linking a sintered body of hydroxyapatite with a substrate surface having a specific functional group via a silane coupler. This enables the substrate surface to be coated with hydroxyapatite. Hydroxyapatite used in this method is obtained by a method of sintering, at high temperature, amorphous hydroxyapatite obtained by various methods. However, powder of hydroxyapatite aggregates during sintering. Consequently, there has been a problem that when this is used for coating a substrate surface, a uniform coat is not formed. Alternatively, when uniformity of a coat is attempted to be increased by dispersing the sintered body, an organic solvent is used as a dispersion medium. Consequently, there has been a problem in terms of safety or the like. In addition, since the silane coupler used is highly hazardous, there has been a problem that this method can be hardly applied to the living body-embedded type implants. Furthermore, in this case, there has been a problem that adhesion with a substrate and abrasion resistance are also poor.

### CITATION LIST

### Patent Literature

PATENT LITERATURE 1: JP-A-62-34559
PATENT LITERATURE 2: JP-A-62-57548
PATENT LITERATURE 3: JP-A-2-255515
PATENT LITERATURE 4: JP-A-11-33106
PATENT LITERATURE 5: JP-A-2005-112848
PATENT LITERATURE 6: JP-A-2004-51952

### SUMMARY OF INVENTION

### Problems to be Solved by the Invention

A problem of the present invention is to provide a bioactive implant that has good bioactivity and can firmly maintain abrasion resistance and adhesion between a metal substrate or a plastic substrate and an apatite layer for an extended period of time.

### Solutions to the Problems

The problem of the present invention is basically solved by the following invention.
1. A bioactive implant having an apatite layer containing at least crystalline calcium apatite fine particles, an aqueous urethane resin, and a self-emulsifiable isocyanate compound, on a surface of a metal substrate or a plastic substrate.
2. The bioactive implant according to the above 1, wherein the apatite layer is plurally present on the metal substrate or the plastic substrate.
3. A manufacturing method of a bioactive implant, including coating a surface of a metal substrate or a plastic substrate with a coating liquid containing at least crystalline calcium apatite fine particles, an aqueous urethane resin, and a self-emulsifiable isocyanate compound to form an apatite layer.

### EFFECTS OF THE INVENTION

According to the present invention, there can be provided a bioactive implant that can have good bioactivity and firmly maintain adhesion between a metal substrate or a plastic substrate and an apatite layer and abrasion resistance for an extended period of time.

### DESCRIPTION OF EMBODIMENTS

A bioactive implant according to the present invention includes an apatite layer containing at least crystalline calcium apatite fine particles, an aqueous urethane resin, and a self-emulsifiable isocyanate compound, on a surface of a metal substrate or a plastic substrate. The apatite layer can be obtained by coating the surface of the metal substrate or the plastic substrate with a coating liquid containing at least crystalline calcium apatite fine particles, an aqueous urethane resin, and a self-emulsifiable isocyanate compound. Each of the components will be described below.

Examples of the crystalline calcium apatite fine particles that can be used in the present invention specifically include fine particles consisting of hydroxyapatite (Ca₁₀(PO₄)₆(OH)₂); fluorapatite (Ca₁₀(PO₄)₆F₂); chlorapatite (Ca₁₀(PO₄)₆Cl₂); carbonate hydroxyapatite (Ca₁₀(PO₄,CO₃)₆(OH)₂) and carbonate fluorapatite (Ca₁₀(PO₄,CO₃)₆F₂) both having a structure in which a portion of a phosphate group contained in these apatites is substituted with a carbonate ion; and a mixture of these. For element compositions of these various calcium apatite fine particles, a ratio of each element is not necessarily fixed according to a stoichiometric ratio represented by a chemical formula. For example, a ratio of a calcium ion may be smaller than a ratio of 10 mol with respect to 6 mol of a phosphate group. That is, a calcium ion may be contained at any ratio between 6 and 10 mol. Furthermore, apatite containing a carbonate group may contain a phosphate group and a carbonate group at any ratio from 1:1 to 1:0. A hydroxyl group and a fluorine ion, or a hydroxyl group and a chlorine ion may also be contained at any ratio from 1:0 to 0:1. Furthermore, magnesium, strontium, sodium, potassium, silicon, iron, or another metal ion may be contained within a range of 1% by mass or less with respect to all elements constituting the calcium apatite fine particles.

There is a preferred size range for the above-described calcium apatite fine particles. The calcium apatite fine particles preferably have a volume average particle size of 0.02 to 5 μm. In particular, a particle size measured using a particle size distribution meter by light scattering and/or a diffraction method while the calcium apatite fine particles are dispersed in a liquid is preferably 0.02 to 5 μm in terms of median diameter as volume average particle size. When the volume average particle size exceeds this range to be larger, abrasion resistance and adhesion between the metal substrate or the plastic substrate and the apatite layer decrease. Consequently, when stress is applied to the surface, the apatite layer is easily peeled off in some cases. When the volume average particle size of the calcium apatite fine particles is smaller than the above-described range, solubility of the apatite layer increases so that the apatite layer is dissolved and disappears into the body fluid for a relatively short period of time in some cases.

Also, when an apatite layer is formed on the surface of the plastic substrate for an implant using, as the calcium apatite fine particles, calcium apatite fine particles in an amorphous state without having crystallinity, solubility of the apatite layer increases too. Consequently, the apatite layer is dissolved and disappears into the body fluid for a relatively short period of time in some cases.

The calcium apatite fine particles that can be used in the present invention need to clearly exhibit crystallinity that is peculiar to apatite. Specifically, the calcium apatite fine particles to be used need to have diffraction peaks which can be clearly determined in wide angle X-ray diffraction measurement. The calcium apatite fine particles having higher crystallinity have lower solubility in the body fluid. This allows the apatite layer to be retained on the surface of the metal substrate or the plastic substrate for an extended period of time, and is thus preferred.

In the crystalline calcium apatite fine particles that can be used in the present invention, characteristic peaks are observed in a range of 10 to 60 degrees for 2θ when wide angle X-ray diffraction measurement is performed in a particulate state. In the present invention, particularly only when each of the diffraction peak from the (002) plane near 26 degrees, the diffraction peak from the (211) plane near 32 degrees, and the diffraction peak from the (300) plane near 33 degrees is clearly observed, the fine particles are considered as having crystallinity. When the diffraction peaks from the (211) plane and the (300) plane are broad and not separated thereby to provide a diffraction pattern that is broad and relatively low in strength, the fine particles are determined to be amorphous calcium apatite that does not have crystallinity.

As crystalline calcium apatite that can be used in the present invention, crystalline calcium apatite prepared by various known techniques can be used. Specifically, various calcium apatites that are available as, for example, a reagent, an industrial chemical, or a food additive grade, a cosmetics grade, a quasi drug grade and a pharmaceutical raw material grade can be used.

Crystalline hydroxyapatite prepared by various manufacturing methods of hydroxyapatite disclosed in literatures below may also be used in the present invention. For example, JP-A-63-159207 discloses a method of mixing calcium carbonate powder and dibasic calcium phosphate (dihydrate) powder to prepare an aqueous slurry, and subsequently grinding and mixing this slurry in a wet grinding mill for allowing reaction to proceed. JP-B-7-115850 discloses a method of preparing hydroxyapatite by performing heating treatment of tricalcium phosphate in an aqueous solution containing an inorganic halide adjusted at pH 7 to 11. JP-A-5-170413 discloses a method of obtaining high-purity fine particles as hydroxyapatite by mixing an aqueous slurry of calcium oxide and/or calcium hydroxide and an aqueous phosphoric acid solution within a range of pH 7 to 12. Hydroxyapatite obtained by the above-described various methods is preferably subjected to hydrothermal treatment or sintering treatment for further increasing their crystallinity. These various methods are each effective as a method for obtaining hydroxyapatite having crystallinity. Therefore, these methods can also be preferably used in the present invention.

Furthermore, as calcium apatite other than hydroxyapatite, various calcium apatites such as fluorapatite, carbonate hydroxyapatite, and carbonate fluorapatite, as described above, may also be preferably used. Examples of a synthesis method of fluorapatite include methods disclosed in JP-A-63-256507, JP-A-5-85709, JP-A-5-85710, JP-A-9-40409, and the like. Examples of a synthesis method of carbonate hydroxyapatite include methods disclosed in JP-A-7-61861, JP-A-8-225312, JP-A-9-218187, JP-A-10-36106, and the like. These calcium apatites may contain, other than calcium, various metal elements such as magnesium and strontium.

When preparing a coating liquid with the above-described crystalline calcium apatite, fine particles of the calcium apatite are preferably used. The calcium apatite fine particles preferably have the volume average particle size previously described. A particularly preferred method as a method for obtaining fine particles is performing wet dispersion treatment of the above-described crystalline calcium apatite in a vehicle. For performing such wet dispersion treatment, various known wet dispersion methods may be employed. The wet dispersion treatment is particularly preferably a wet dispersion method using media. Specifically, media such as glass beads, alumina beads, or other ceramic beads are usually added to a vehicle to which the crystalline calcium apatite has been introduced, and the mixture is shaken or agitated. Fine particles of the calcium apatite are obtained by allowing the crystalline calcium apatite particles and the beads to mechanically collide with each other in this manner. When a small amount of calcium apatite is treated in a batchwise manner, shaking is performed using a paint conditioner for several hours. Thus, wet dispersion treatment can be performed. When a relatively large amount of a sample is used for the treatment, a media disperser such as a ball mill and a Dyno mill may be used for performing wet dispersion treatment. The media disperser may be plurally arranged in series to perform wet dispersion treatment in one pass. Alternatively, one media disperser may also be preferably used to perform wet dispersion treatment by repeating the treatment multiple times.

The vehicle for dispersing crystalline calcium apatite is most preferably water. Furthermore, various solvents having miscibility with water may also be used as long as the added amount of solvents is less than 20% by mass with respect to water. Examples of the solvents include alcohols such as methanol, ethanol, and propanol; cyclic ethers such as 1,3-dioxolane, 1,4-dioxane, and tetrahydrofuran; ketones such as acetone and methyl ethyl ketone; and polar solvents such as acetonitrile dimethylformamide.

When performing wet dispersion treatment by utilizing the above-described media, ceramic beads are preferably used as the media. In particular, it is preferred to inhibit the beads from being brought in contact with calcium apatite to be ground so that beads-derived impurities mix in a calcium apatite dispersion. Specific examples of the ceramic beads that can be used for such a purpose include zirconia-containing ceramic beads such as ZrO, cubic zirconia, yttrium-stabilized zirconia, and zirconia-toughened alumina; synthetic diamond; and silicon nitride beads. The media has an average diameter of preferably 0.01 to 10 mm, and more preferably 0.1 to 5 mm. The condition of the wet dispersion treatment using the media disperser with such media is treatment at room temperature as usually performed. The treatment time, temperature, and the like are not particularly limited. Furthermore, one pass is enough in some cases. However, treatment with approximately 2 to 7 passes is preferred, because a dispersion of calcium apatite fine particles having a narrower particle size distribution and excellent dispersion stability is obtained.

Next, the aqueous urethane resin will be described.

The aqueous urethane resin used in the present invention is preferably a water-dispersible polyurethane emulsion. An example thereof includes a self-emulsifiable polyurethane resin. The self-emulsifiable polyurethane resin is preferably an urethane resin having in the polyurethane structure a hydrophilic group such as a sulfonic acid group, a carboxy group, a hydroxyl group, and a polyethyleneoxy group. Various commercially available water-dispersible polyurethane emulsions are preferably used.

Various polyurethane resins are conventionally used in medical applications. For example, JP-T-2006-516467 discloses a structure of a bone implant containing a biodegradable polyurethane resin and hydroxyapatite. The biodegradable polyurethane resin is gradually degraded and absorbed in the living body. For this reason, even when an apatite layer containing such a biodegradable polyurethane resin and hydroxyapatite was attempted to be applied to the present invention, it was difficult to provide a bioactive implant that can firmly maintain adhesion between the plastic substrate and the apatite layer and abrasion resistance for an extended period of time. Therefore, the biodegradable polyurethane resin is not contained in the aqueous urethane resin used in the present invention.

The water-dispersible polyurethane emulsion will be described in further detail. The water-dispersible polyurethane emulsion is a polyurethane resin that is stably dispersed in water. The polyurethane resin has a particle size of preferably 1 μm or less, and further preferably 0.5 μm or less, in terms of volume average particle size. The polyurethane resin having such a volume average particle size increases an ability of allowing the metal substrate and the plastic substrate to adhere with the apatite layer, and thus can be most preferably used. The lower limit of the particle size is preferably 0.02 μm or more. There are various manufacturing methods for forming the polyurethane resin in a form of being stably dispersed in water. As the water-dispersible polyurethane emulsion that can be preferably used in the present invention, any resin emulsion having a polyurethane structure can be essentially used regardless of the manufacturing method thereof As described herein, the resin emulsion having a polyurethane structure is a resin emulsion having a polyurethane structure obtained by addition-polymerization between organic diisocyanate or polyisocyanate and organic diol or polyol. Examples of the organic diisocyanate or polyisocyanate include: as aromatic diisocyanate, for example, toluene diisocyanate, tetramethyl xylylene diisocyanate, diphenyl methane diisocyanate, m-xylylene diisocyanate, and naphthalene diisocyanate; as C2 to C12 aliphatic diisocyanate, for example, hexamethylene diisocyanate, 2,2,4-trimethyl hexane diisocyanate, and lysine diisocyanate; and, as C4 to C18 alicyclic diisocyanate, for example, 1,4-cyclohexane diisocyanate, isophorone diisocyanate, 4,4′-dicyclohexylmethane diisocyanate, methylcyclohexane diisocyanate, and isopropylidene dicyclohexyl-4,4′-diisocyanate. Furthermore, as modified products of all these diisocyanates, there are included carbodiimide, uretdione, biuret and/or isocyanurate-modified products. Furthermore, for stably dispersing a formed polyurethane resin in water, there is included, as a preferred example, a method of obtaining a water-dispersible polyurethane emulsion by using polyisocyanates including an alkyleneoxy group bonded thereto as the self-emulsifiable isocyanate.

Examples of the above-described organic diol or polyol include: as aliphatic diol, for example, ethylene glycol, propylene glycol, 1,4-butanediol, glycerin, and trimethylol propane; as aromatic diol, for example, bisphenol A; or as polyether polyol, for example, polyethylene glycol, polypropylene glycol, polyoxyethylene oxypropylene (block or random) glycol, and polyoxytetramethylene glycol. Alternatively, another example of the polyol includes polyester polyol. Specific examples include: as aliphatic diol, for example, ethylene glycol, propylene glycol, 1,4-butanediol, glycerin, and trimethylol propane; as aromatic diol, for example, polyester polyol obtained by condensation between bisphenol A or the like and dicarboxylic acid (succinic acid, adipic acid, sebacic acid, terephthalic acid, isophthalic acid, and the like), and polylactone polyol such as polycaprolactone polyol and polyvalerolactone polyol. Furthermore, another preferred example thereof includes polycarbonate diol such as polybutylene carbonate diol and polyhexamethylene carbonate diol. In addition, organic diol having a polyalkyleneoxy group is preferably used as a polyol component in order to stably disperse a formed polyurethane resin in water (for example, U.S. Pat. No. 3,905,929 and U.S. Pat. No. 5,043,381).

An example of a method for stably dispersing the above-described polyurethane resin obtained by addition polymerization between the organic diisocyanate or polyisocyanate and the organic diol or polyol includes the manufacturing method of a water-dispersible polyurethane emulsion disclosed in JP-B-53-38760 and JP-B-63-8141. In this method, a terminal isocyanate group-containing urethane prepolymer having in its molecule an anionic group such as a carboxyl group is neutralized with tertiary amine to provide a state of being emulsifiable in water. Subsequently, the obtained product is subjected to chain elongation to manufacture a water-dispersible polyurethane emulsion. Another example includes the manufacturing method of a water-dispersible polyurethane emulsion disclosed in Japanese Patent Application No. 3-327393, JP-A-6-93068, and the like. In this method, a polyurethane resin having in its molecule an anionic group such as a carboxy group is synthesized. Subsequently, the obtained product is neutralized with amines to obtain a water-dispersible polyurethane emulsion that has become emulsifiable in water.

The water-dispersible polyurethane emulsion can also be synthesized in a state of being emulsified in water as described above. Alternatively, it is also preferred to perform polymerization with an organic solvent such as ketones and ethers having miscibility with water and subsequently add water to distill away the organic solvent for converting into a water-dispersible polyurethane emulsion.

As the aqueous urethane resin that can be used in the present invention, the water-dispersible polyurethane emulsion obtained by the above-described various methods and materials can be preferably used. Examples thereof may include a polyurethane emulsion represented by trade name HYDRAN available from DIC Corporation, and an aqueous urethane resin represented by trade name PERMARIN, UPRENE, UCOAT, and the like available from Sanyo Chemical Industries, Ltd.

There is a preferred range for the ratio between the aqueous urethane resin and the calcium apatite fine particles that are used in the present invention. In the present invention, the dry solid mass ratio between the calcium apatite fine particles and the aqueous urethane resin that are contained in the apatite layer is preferably 1:0.1 to 1:1.5. Furthermore, the dry solid mass ratio between the calcium apatite fine particles and the aqueous urethane resin that are contained in the coating liquid used when forming the apatite layer in the present invention is preferably 1:0.1 to 1:1.5. When the dry solid mass ratio of the aqueous urethane resin to the apatite as 1 is less than 0.1, adhesion and abrasion resistance at the interface between the apatite layer and the substrate sometimes deteriorate. Consequently, stress such as friction sometimes causes the apatite layer to easily peel off. Also, when the dry solid mass ratio of the aqueous urethane resin to the apatite as 1 is more than 1.5, the surface of the calcium apatite fine particles is covered with the aqueous urethane resin in the apatite layer formed on the substrate surface. Therefore, bioactivity may decreases.

By coating the surface of the metal substrate or the plastic substrate described later with the coating liquid containing a combination of the above-described aqueous urethane resin and calcium apatite fine particles, the calcium apatite fine particles can be firmly bonded to the substrate surface. In the present invention, for a purpose of retaining the apatite layer on the substrate surface for an extended period of time while further enhancing adhesion and abrasion resistance, the apatite layer possessed by the bioactive implant according to the present invention further contains a self-emulsifiable isocyanate compound. The self-emulsifiable isocyanate compound that is used in the present invention is a compound having a repeating unit of ethylene oxide, and further having two or more isocyanate groups. An example of such a compound includes the self-emulsifiable isocyanate disclosed in JP-B-55-7472 (U.S. Pat. No. 3,996,154), JP-A-5-222150 (U.S. Pat. No. 5,252,696), JP-A-9-71720, JP-A-9-328654, JP-A-10-60073, and the like. A specific preferred example includes polyisocyanate having in its molecule an isocyanurate structure having a cyclic trimer backbone formed from aliphatic or alicyclic diisocyanate. Another example includes a polyisocyanate compound having a structure obtained by using, as base polyisocyanate, polyisocyanate having in its molecule a biuret structure or a urethane structure and adding polyethylene glycol having one etherified terminal or the like to only part of the polyisocyanate group. A synthesis method of the isocyanate compound having such a structure is described in the above-described various bulletins. Furthermore, as the isocyanate compound having such a structure, a product including, as base polyisocyanate, polyisocyanate obtained by cyclotrimerization with hexamethylene diisocyanate or the like as a starting material is commercially available. For example, a self-emulsifiable isocyanate compound under trade name Duranate commercially available from Asahi Chemical Industry Co., Ltd. may be used. These self-emulsifiable isocyanate compounds have high hydrophilicity. Consequently, the use of these self-emulsifiable isocyanate compounds on the surface of the bioactive implant together with the calcium apatite and the aqueous urethane resin can maintain the surface in a further highly hydrophilic state and further increase bioactivity. Therefore, the self-emulsifiable isocyanate compound can be preferably used.

There are preferred ranges for both the ratio of the self-emulsifiable isocyanate compound contained in the apatite layer in the present invention and the self-emulsifiable isocyanate compound in the coating liquid used when forming the apatite layer in the present invention. In both cases, the ratio of the self-emulsifiable isocyanate compound with respect to the dry solid mass of the used aqueous urethane resin is preferably 1 to 50% by mass, and further preferably 5 to 40% by mass.

The apatite layer containing the above-described components is obtained by applying a coating liquid containing these components on the surface of the metal substrate or the plastic substrate. The coating liquid can contain various surfactants as necessary. Examples of anionic surfactants that can be used in the present invention include: higher fatty acid salts such as sodium laurate, sodium stearate, and sodium oleate; alkyl sulfate salts such as sodium dioctyl sulfosuccinate, sodium lauryl sulfate, and sodium stearyl sulfate; higher alcohol sulfate ester salts such as sodium octyl alcohol sulfate, sodium lauryl alcohol sulfate, and ammonium lauryl alcohol sulfate; aliphatic alcohol sulfate ester salts such as sodium acetyl alcohol sulfate; alkyl benzene sulfonate salts such as sodium dodecyl benzene sulfonate; alkyl naphthalene sulfonate salts such as sodium butyl naphthalene sulfonate and sodium isopropyl naphthalene sulfonate; alkyl diphenyl ether disulfonate salts such as sodium alkyl diphenyl ether disulfonate; alkyl phosphate ester salts such as sodium lauryl phosphate and sodium stearyl phosphate; polyethylene oxide adducts of alkyl ether sulfate salt such as a polyethylene oxide adduct of sodium lauryl ether sulfate, a polyethylene oxide adduct of ammonium lauryl ether sulfate, and a polyethylene oxide adduct of triethanolamine lauryl ether sulfate; polyethylene oxide adducts of alkyl phenyl ether sulfate salt such as a polyethylene oxide adduct of sodium nonylphenyl ether sulfate; polyethylene oxide adducts of alkyl ether phosphate salt such as a polyethylene oxide adduct of sodium lauryl ether phosphate; and polyethylene oxide adducts of alkyl phenyl ether phosphate salt such as a polyethylene oxide adduct of sodium nonylphenyl ether phosphate.

The nonionic surfactant that can be used in the present invention is preferably polyethylene oxide alkyl ether and polyethylene oxide alkyl phenyl ether in which an alkyl group, a phenyl group, and an alkyl-substituted phenyl group are bonded to polyethylene oxides having various chain lengths. Among these, sorbitan monoalkylate derivatives known as trade name TWEEN 20, TWEEN 40, TWEEN 60, and TWEEN 80 are suitable.

When the coating liquid contains the surfactants in the present invention, there is a preferred range. The content rate of the surfactants, in terms of solid content mass ratio with respect to the calcium apatite fine particles contained in the coating liquid, is preferably 5% by mass or less, and further preferably 3% by mass or less.

In the present invention, the apatite layer may further contain various water-soluble polymers as necessary. Examples of the water-soluble polymers include gelatine, gelatine derivatives (for example, phthalated gelatine), hydroxyethyl cellulose, carboxymethyl cellulose, methyl cellulose, hydroxypropyl methyl cellulose, ethyl hydroxyethyl cellulose, polyvinyl pyrrolidone, polyethylene oxide, xanthan, cationic hydroxyethyl cellulose, polyacrylic acid, sodium polyacrylate, polyvinyl alcohol, polyacrylamide, polyvinyl pyrrolidone, starch, and various modified starches (for example, phosphoric acid modified-starch). The amount of the water-soluble polymers with respect to the aqueous urethane resin is preferably an amount that does not exceed the dry solid amount of the aqueous urethane resin. When the content of the water-soluble polymers is higher than that of the aqueous urethane resin, adhesion and abrasion resistance of the apatite layer deteriorate in some cases.

Examples of the metal substrate possessed by the bioactive implant according to the present invention include various medical-grade metal substrates that can be used for implants. In particular, a metal substrate containing titanium such as pure titanium, titanium-aluminum-vanadium alloy (such as Ti-6Al-4V), titanium-aluminum-niobium alloy (such as Ti-6Al-7Nb), and nickel-titanium alloy can be particularly preferably used.

Preferred examples of the plastic substrate possessed by the bioactive implant according to the present invention include various polyesters; polycarbonate; and PEEK (polyether ether ketone), PEKK (polyether ketone ketone), and the like as aromatic polyketone, which are of a medical-grade and can be used for implants. Furthermore, carbon fiber reinforced plastic, which includes these plastics as a substrate and carbon fiber incorporated in the substrate, is also preferably used.

The above-described metal substrate and plastic substrate are preferably subjected to coating in a state of having been previously molded into a shape as an implant to be applied. Examples of a specific shape of the substrate include various shapes such as rod-like, block-like, flat plate-like, string-like, thread-like, fibrous, coiled, or porous body. Particularly for maintaining good bondability with bones, a coating method using a substrate having a roughed surface can also be preferably used. Alternatively preferred example includes a substrate having a structure in which voids and porous diaphragms are disposed like in the implant used as an artificial intervertebral spacer to provide a structure of facilitating intrusion of living bones into the implant. Also, the metal substrate and plastic substrate to be used in the present invention may have been subjected to hydrophilization treatment on their surfaces. Examples of such hydrophilization processing include corona discharge treatment, flame treatment, plasma treatment, and ultraviolet irradiation treatment. As further hydrophilization processing, the metal substrate and plastic substrate may have a base layer. As the base layer, a layer containing a hydrophilic resin is effective. Examples of the hydrophilic resin include gelatine, gelatine derivatives (for example, phthalated gelatine), hydroxyethyl cellulose, carboxymethyl cellulose, methylcellulose, hydroxypropyl methyl cellulose, ethyl hydroxyethyl cellulose, polyvinyl pyrrolidone, polyethylene oxides, xanthan, cationic hydroxyethyl cellulose, polyvinyl alcohol, and polyacrylamide.

As a coating method that can be preferably used when disposing the apatite layer on the metal or plastic substrate, the most suitable coating method can be selected depending on the type of a substrate. Specifically, a spray coating technique and impregnation (dip coating technique) in which a substrate is dipped in a coating liquid can also be preferably used.

The condition when coating the substrate in the above-described methods is that heat drying is performed at a temperature of preferably at least 40° C. or higher, and further preferably 70° C. or higher. This enables further increase in adhesion between the formed apatite layer and the substrate, and dramatic improvement in adhesion and abrasion resistance of the apatite layer. After the coating and drying, heating treatment is preferably further performed at a temperature in a range of 30 to 90° C. for several hours to several days.

In the present invention, when a necessary thickness cannot be obtained by one coating, coating and drying can be repeated multiple times to form the apatite layer. The thickness of the apatite layer prepared in this manner is preferably 0.1 to 50 μm. When the apatite layer has a thickness of less than 0.1 μm, the effects of the present invention may not be observed. On the other hand, when the apatite layer has a thickness of more than 50 μm, cracks may be caused on the surface when the thickness of the apatite layer becomes non-uniform during coating and drying.

Preferably, the apatite layer is plurally disposed on the surface of the metal substrate or the plastic substrate. In this case, the plurality of apatite layers having difference structures is preferably stacked on each other. The apatite layers having different structures may be formed by stacking a plurality of apatite layers containing different types of calcium apatite. Alternatively, the apatite layers having different structures may be formed by stacking a plurality of apatite layers having different ratios between calcium apatite and the aqueous urethane resin constituting the apatite layer.

As an example of a preferred embodiment, an apatite layer containing fluorapatite is firstly formed on a plastic substrate, and an apatite layer containing hydroxyapatite is formed on the formed fluorapatite-containing apatite layer, so that the apatite layers having low solubility and good acid resistance are firmly fixed on the surface of the plastic substrate. Consequently, it can be expected that connection between the obtained implant and bones is firmly retained for an extended period of time. Furthermore, an apatite layer containing hydroxyapatite, carbonate hydroxyapatite, or the like is formed on the outermost surface of the implant. Consequently, since calcium apatite is facilitated to be reabsorbed and substituted with the living bone in the living body, it is expected that connection between the implant surface and the living bone becomes firm.

Alternatively, when forming a plurality of apatite layers having various different ratios between the aqueous urethane resin and the calcium apatite fine particles on the surface of the substrate, it is preferred that an apatite layer having a high ratio of the aqueous urethane resin is formed on the substrate surface, and on the formed apatite layer, an apatite layer having a relatively low ratio of the aqueous urethane resin is formed. Accordingly, there can be formed an implant in which the outermost surface of the implant has higher bioactivity while the strength of the adhesive surface between the substrate and the apatite layer is higher.

In the present invention, the implant having the above-described apatite layers is preferably washed prior to use of the implant, so that water-soluble components (soluble substances such as various water-soluble salts and surfactants) are removed. In particular, the washing is preferably performed with pure water that does not contain impurities, preferably with pure water at 70° C. or higher, for sufficiently washing the surface and inside of the implant. Subsequently, sterilization treatment is preferably further performed using an autoclave, an ethylene oxide sterilizer, an electron beam irradiation apparatus, a gamma irradiation apparatus, or the like.

Hereinafter, the present invention will be described in further detail by referring to examples. However, the present invention is not limited to these examples. It is noted that percentages in the examples are on a mass basis unless otherwise stated.

### EXAMPLES

### Example 1

### (Preparation of Dispersion Liquid of Crystalline Hydroxyapatite)

As calcium apatite, medical-grade hydroxyapatite HAP-100 available from Taihei Chemical Industrial Co. Ltd. was used. The calcium apatite was subjected to wide angle X-ray diffraction measurement. As a result, sharp diffraction peaks were clearly observed from (002) plane near 26 degrees, (211) plane near 32 degrees, and (300) plane near 33 degrees, which are characteristic of hydroxyapatite. This demonstrated that the calcium apatite had crystallinity. The calcium apatite was subjected to wet dispersion treatment by a bead mill method in the following manner. That is, 20 g of the above-described hydroxyapatite was poured into a 0.2 L polypropylene container. Into this, 100 g of ion exchanged water and 200 g of zirconia beads having a particle size of 0.3 mm were added, and the container was sealed. Shaking treatment was performed using a paint conditioner for 6 hours. Thereafter, the zirconia beads were separated from the dispersion liquid through a filter cloth. The size of the hydroxyapatite fine particles in a dispersion liquid 1 obtained was measured using a light scattering diffraction particle size distribution meter (particle size distribution meter LA-920 manufactured by Horiba, Ltd.). The obtained volume average particle size was 1.36 μm in terms of median diameter, and 95% by mass of the particles fell within a range of 0.4 to 2.6 μm. A relatively narrow particle size distribution was exhibited.

### (Preparation of Coating Liquid 1 Containing Crystalline Calcium Apatite)

Into 100 g of the above-described dispersion liquid 1 of hydroxyapatite fine particles (solid content concentration: 16.7% by mass), 15 g of HYDRAN AP-40F (solid content concentration: 22.5% by mass, volume average particle size: 0.15 μm) manufactured by DIC Corporation were added as an aqueous urethane resin. Furthermore, 0.85 g of Duranate WB40-80 (solid content concentration: 80% by mass) manufactured by Asahi Chemical Industry Co., Ltd. were added as a self-emulsifiable isocyanate compound. Water was further added to adjust the solid content concentration to 8% by mass. The resultant product was stirred at room temperature for one hour to prepare a coating liquid 1 to be used in the present invention.

### (Preparation of Bioactive Implant Model)

As a model of a metal substrate and a plastic substrate to be used in an implant, a pure titanium plate and a polyester film (LUMIRROR manufactured by Toray Industries, Inc.) having a thickness of 100 μm were prepared respectively. These were coated with the above-described coating liquid 1 using a doctor bar such that the coating amount of the coating liquid 1 containing hydroxyapatite fine particles became 36 g per m². The coated substrates were dried at a temperature of 80° C. using a dryer. The apatite layer formed on each of the titanium plate and the polyester film was further heated for 24 hours in a drying oven adjusted at 40° C. Thereafter, the heated apatite layer was immersed in boiling pure water for one minute for washing. The surface and cross section of the apatite layer were observed through a scanning electron microscope. An apatite layer, in which fusiform fine particles having a length of about 60 nm as primary particles of hydroxyapatite was densely accumulated, had been formed. The apatite layer was demonstrated to have a thickness of about 1.5 μm.

### (Adhesion and Abrasion Resistance Test of Apatite Layer)

The apatite layer formed on the surface of each of the above-described titanium plate and polyester film was evaluated in the following manner. First, the following test was performed as evaluation for adhesion and abrasion resistance using a fastness to rubbing tester (RT-300 manufactured by Daiei Kagaku Seiki Mfg. Co., Ltd.). That is, the surface of the apatite layer was rubbed for 6 hours (about 10,000 strokes) with 30 strokes/min in a state where a load of 300 g was applied to a planar friction block (a 2 cm square on the bottom) while maintaining a state where the surface of the apatite layer was wet with water. As a result, no change was observed in the rubbed portion of the apatite layer by visual observations. That is, it was demonstrated that good adhesion and abrasion resistance were exhibited for both substrates of the titanium plate and the polyester film.

(Evaluation for Bioactivity with Pseudo Body Fluid)

Both of the substrates on which the above-described apatite layers were formed were immersed in a pseudo body fluid which mimicks human plasma (an inorganic salt-containing aqueous solution (1) having a composition disclosed in PATENT LITERATURE 4) at 37° C. for 7 days. Observations through a scanning electron microscope demonstrated that this caused a further thick and smooth apatite layer to be formed on the surface of the apatite layer. The apatite layer formed with the pseudo body fluid had a thickness of about 5 μm). As comparison, the above-described titanium plate and polyester film were immersed in the pseudo body fluid together with the above-described substrates to observe the appearance of the surface. However, in this case, an apatite layer was not formed on the surfaces of the titanium plate and polyester film. This clearly demonstrated that the bioactive implant model, on which the apatite layer was formed by applying the above-described coating liquid 1, exhibited excellent bioactivity.

### (Formation of Apatite Layer to Artificial Ligament)

An artificial ligament selected as one of the living body-embedded type implants was coated with calcium apatite. The apatite layer was evaluated for bioactivity, adhesion, and abrasion resistance. As a plastic substrate, “Telos artificial ligament (polyester cord)” manufactured by and available from Ai-Medic Co., Ltd. was used. Both ends of this artificial ligament, corresponding to a portion entering a femoral opening and a portion entering a tibial opening, were immersed in the above-described coating liquid 1 containing crystalline calcium apatite. The removed artificial ligament was left to stand in a drying oven at 70° C. for 3 hours to perform drying and heating treatment. An apatite layer was formed on the surface of the polyester cord. The artificial ligament on which the apatite layer was formed in this manner was repeatedly washed with hot water heated to 70° C. Thereafter, sterilization treatment was performed with ethylene oxide gas. It is noted that the cross section was separately observed through a scanning electron microscope. As a result, the apatite layer had a thickness of about 2 μm.

### (Evaluation for Adhesion and Abrasion Resistance of Apatite Layer on Artificial Ligament Surface)

The artificial ligament having the apatite layer prepared as described above was repeatedly rubbed using a fastness to rubbing tester in the same method as above. Any change was not observed in the apatite layer. The artificial ligament including the apatite layer formed thereon was further immersed and stored in a physiological salt solution adjusted to pH 6 at 40° C. for 3 months. Thereafter, the artificial ligament was repeatedly rubbed using a fastness to rubbing tester in the same method. Any change was not observed in the apatite layer. This demonstrated that good adhesion and abrasion resistance was maintained. Also, the artificial ligament having the apatite layer prepared as above was immersed in a pseudo body fluid at 37° C. for 7 days in the same method as the above-described evaluation for bioactivity. As a result, a calcium apatite layer due to the pseudo body fluid was further formed in a thickness of about 2 μm on the apatite layer surface. As a result, it was clearly demonstrated that the apatite layer exhibited excellent bioactivity.

### Comparative Example 1

A coating liquid 2, which does not contain Duranate WB40-80 used as a self-emulsifiable isocyanate compound in the above-described coating liquid 1 containing crystalline calcium apatite prepared in Example 1, was prepared. A surface of an artificial ligament was coated in the same method as Example 1, except that the coating liquid 2 was used in place of the coating liquid 1, to form an apatite layer. Washing and sterilization treatment was also performed in the same method as Example 1. The artificial ligament including the comparative apatite layer prepared as above was immersed and stored in a physiological salt solution adjusted to pH 6 at 40° C. for 3 months. Thereafter, the artificial ligament was repeatedly rubbed using a fastness to rubbing tester in the same method as Example 1. As a result, the apatite layer was easily peeled off from the surface of the artificial ligament. This demonstrated that adhesion was lacking.

### Comparative Example 2

A coating liquid 3 was prepared by adding hydrophobic isophorone diisocyanate which was not self-emulsifiable, in place of the self-emulsifiable isocyanate compound in the coating liquid 1 containing crystalline calcium apatite prepared in Example 1. An artificial ligament was coated in the same method as Example 1, except that the coating liquid 3 was used in place of the coating liquid 1. Washing and sterilization treatment was also performed in the same method as Example 1. However, the coating liquid 3 did not uniformly stick to the surface of the artificial ligament. Consequently, portions where an apatite layer was not formed appeared on the polyester surface. Even when the artificial ligament was immersed in a pseudo body fluid, an apatite layer was not formed on such portions. For this reason, there was obtained a result that this apatite layer had poor bioactivity.

### Comparative Example 3

An amorphous hydroxyapatite was used which was synthesized in accordance with the method disclosed in JP-A-5-170413, in place of hydroxyapatite (HAP-100) having crystallinity used in Example 1. The amorphous hydroxyapatite was subjected to wet dispersion treatment in the same method as Example 1 to obtain a dispersion liquid 2 containing amorphous hydroxyapatite fine particles having a volume average particle size of 1.1 μm. A coating liquid 4 was prepared in the same method as Example 1, except that the dispersion liquid 2 was used in place of the dispersion liquid 1 which was used for preparing the coating liquid 1 containing crystalline calcium apatite of Example 1. Then, an apatite layer was formed on the surface of an artificial ligament with the coating liquid 4 in the same method as Example 1. Washing and sterilization treatment was also performed in the same method as Example 1. The artificial ligament including the apatite layer prepared as above was immersed and stored in a physiological salt solution adjusted to pH 6 at 40° C. for 3 months. Thereafter, the artificial ligament was repeatedly rubbed using a fastness to rubbing tester in the same method as Example 1. As a result, the apatite layer was easily peeled off from the surface of the artificial ligament. This demonstrated that adhesion was lacking.

### Example 2

### (Preparation of Dispersion Liquid of Crystalline Fluorapatite)

Fluorapatite FAP available from Taihei Chemical Industrial Co. Ltd., which is used as calcium apatite in place of HAP-100 of Example 1, was subjected to wet dispersion treatment by a bead mill method in the same manner. The used FAP was previously confirmed to be crystalline by wide angle X-ray diffraction measurement. With a dispersion liquid 3 obtained, the size of the dispersing fluorapatite fine particles was measured using a light scattering diffraction particle size distribution meter (particle size distribution meter LA-920 manufactured by Horiba, Ltd.). The obtained volume average particle size was 1.5 μm, and exhibited a relatively narrow particle size distribution.

### (Preparation of Coating Liquid 5 Containing Crystalline Calcium Apatite)

Into 100 g of the above-described dispersion liquid 3 of fluorapatite fine particles (solid content concentration: 16.7% by mass), 30 g of HYDRAN AP-40F (solid content concentration: 22.5% by mass, volume average particle size: 0.15 μm) manufactured by DIC Corporation were added as an aqueous urethane resin. Furthermore, 1.5 g of Duranate WB40-80 (solid content concentration: 80% by mass) manufactured by Asahi Chemical Industry Co., Ltd. were added as a self-emulsifiable isocyanate compound. Water was further added to adjust the solid content concentration to 8% by mass. Subsequently, the resultant product was stirred at room temperature for one hour to prepare a coating liquid 5 to be used in the present invention.

### (Preparation of Bioactive Implant Model)

A pure titanium plate and a PEEK film (FS-1100C manufactured by Sumitomo Bakelite Co., Ltd.) having a thickness of 100 μm selected as a substrate model to be used in an implant were coated with the coating liquid 5 prepared as described above using a doctor bar, such that the coating amount of the coating liquid containing fluorapatite fine particles became 18 g per m². Drying was performed using a dryer. The apatite layer formed on the pure titanium plate and the PEEK film was further heated in a drying oven adjusted to 40° C. for 24 hours. The surface and cross section of the apatite layer was observed through a scanning electron microscope. As a result, it was demonstrated that the formed apatite layer included densely accumulated fluorapatite fine particles and had a thickness of about 0.8 μm. Furthermore, the coating liquid 1 containing crystalline hydroxyapatite prepared in Example 1 was applied on the top of the formed apatite layer using a doctor bar, such that the coating amount of the coating liquid containing hydroxyapatite fine particles became 18 g per m², and then dried using a dryer. Accordingly, an apatite layer (thickness: about 0.8 μm) containing hydroxyapatite was formed on the apatite layer containing fluorapatite. Thereafter, heating treatment was performed in the same method as Example 1 in a drying oven adjusted to 40° C. for 24 hours. Thereafter, immersion in boiling pure water for one minute was performed for washing to prepare a bioactive implant model.

### (Adhesion and Abrasion Resistance Test of Apatite Layer)

The apatite layer formed on the surface of each of the above-described titanium plate and PEEK film substrates was evaluated in the following manner. First, the following test was performed as evaluation for adhesion and abrasion resistance using a fastness to rubbing tester (RT-300 manufactured by Daiei Kagaku Seiki Mfg. Co., Ltd.). That is, the apatite layer surface was rubbed for 6 hours (about 10,000 strokes) with 30 strokes/min in a state where a load of 600 g was applied to a planar friction block (a 2 cm square on the bottom) while maintaining a state where the apatite layer surface was wet with water. As a result, no change was observed in the rubbed portion of the apatite layer by visual observations. It was demonstrated that good adhesion and abrasion resistance were exhibited to both substrates of the titanium plate and the PEEK film.

(Evaluation for Bioactivity with Pseudo Body Fluid)

Each of the above-described titanium plate and PEEK film on which the apatite layers were formed was immersed in a pseudo body fluid at 37° C. for 7 days in the same method as the above-described evaluation for bioactivity. As a result, it was confirmed that a calcium apatite layer due to the pseudo body fluid was further formed in a thickness of about 2 μm on the surface of the apatite layer. This demonstrated that high bioactivity was exhibited.

### (Formation of Apatite Layer to Spinal Cage)

As one of biological implants, a spinal cage used as an intervertebral spacer for spinal fusion was selected. This spinal cage was coated with calcium apatite. The apatite layer was evaluated for bioactivity, adhesion, and abrasion resistance. As the spinal cage, an implant made of “sterilized CAPSTONE PEEK” PEEK manufactured by and available from Medtronic Sofamor Danek, Co., Ltd. was used. This spinal cage was immersed in the above-described coating liquid 5 containing crystalline fluorapatite. The removed spinal cage was left to stand in a drying oven at 70° C. for 3 hours to perform drying and heating treatment. Subsequently, the coating liquid 1 containing crystalline hydroxyapatite prepared in Example 1 was applied in the same method. Drying and heating treatment was performed to prepare a spinal cage on which an apatite layer containing hydroxyapatite was formed on an apatite layer containing fluorapatite. The spinal cage having the stacked apatite layers formed thereon in this manner was repeatedly washed with hot water heated to 70° C. Thereafter, sterilization treatment was performed with ethylene oxide gas. The prepared spinal cage having the apatite layers was immersed in a pseudo body fluid for 7 days. As a result, it was confirmed that a calcium apatite layer due to the pseudo body fluid was further formed in a thickness of about 2 μm on the surface of the apatite layer. It became clear that excellent bioactivity was exhibited.

### (Evaluation for Adhesion and Abrasion Resistance of Apatite Layer on Spinal Cage Surface)

The spinal cage having the apatite layers prepared as described above was immersed and stored in a physiological salt solution adjusted to pH 6, at 50° C. for 3 months. Thereafter, the surface of the apatite layer was strongly rubbed in a repeated manner using nonwoven fabric. Any change was not observed in the apatite layer. It was confirmed that good adhesion and abrasion resistance were maintained.

### Example 3

### (Preparation of Coating Liquid 6 Containing Crystalline Calcium Apatite)

Into 100 g of the dispersion liquid 1 of hydroxyapatite fine particles (solid content concentration: 16.7% by mass) prepared in Example 1, 50 g of HYDRAN AP-40F (solid content concentration: 22.5% by mass, volume average particle size: 0.15 μm) manufactured by DIC Corporation were added as an aqueous urethane resin. Furthermore, 3 g of Duranate WB40-80 (solid content concentration: 80% by mass) manufactured by Asahi Chemical Industry Co., Ltd. were added as a self-emulsifiable isocyanate compound. Water was further added to adjust the solid content concentration to 10% by mass. Subsequently, the resultant product was stirred at room temperature for one hour to prepare a coating liquid 6 to be used in the present invention.

### (Formation of Apatite Layer to Artificial Tooth Root)

As one of biological implants, an artificial tooth root used as a dental implant was selected. This artificial tooth root was coated with calcium apatite. The apatite layer was evaluated for bioactivity, adhesion, and abrasion resistance. As the artificial tooth root, “POI one-piece implant (an implant made of titanium)” manufactured by and available from KYOCERA Medical Corporation was used. Only the screw portion of this artificial tooth root was immersed in the above-described coating liquid 6 containing crystalline calcium apatite. The removed artificial tooth root was left to stand in a drying oven at 70° C. for 10 hours to perform drying and heating treatment. In this manner, the artificial tooth root including the embedded screw portion on which the apatite layer was applied was prepared. Furthermore, the artificial tooth root was repeatedly washed with hot water heated to 70° C., and then subjected to sterilization treatment with ethylene oxide gas.

### (Evaluation for Adhesion and Abrasion Resistance of Apatite Layer on Artificial Tooth Root Surface)

The artificial tooth root having the apatite layer prepared as described above was immersed and stored in a physiological salt solution adjusted to pH 5, at 50° C. for 6 months. Thereafter, the surface was strongly rubbed with a hand using nonwoven fabric. Any change was not observed in the apatite layer. It was confirmed that good adhesion and abrasion resistance were maintained.

(Evaluation for Bioactivity with Pseudo Body Fluid)

The artificial tooth root having the apatite layer prepared as described above was immersed in a pseudo body fluid for 7 days. It was confirmed that a calcium apatite layer due to the pseudo body fluid was further formed in a thickness of about 2 μm on the surface of the apatite layer. It became clear that excellent bioactivity was exhibited.

### INDUSTRIAL APPLICABILITY

An apatite layer containing crystalline hydroxyapatite fine particles, an aqueous urethane resin, and a self-emulsifiable isocyanate compound has a surface that is hydrophilic and excellent in water resistance, adhesion, and abrasion resistance. Therefore, various hydrophilic materials having compatibility with the living body can be provided by performing surface treatment on various metal plates, films, and fibers, other than a bioactive implant.

1. A bioactive implant having an apatite layer containing at least crystalline calcium apatite fine particles, an aqueous urethane resin, and a self-emulsifiable isocyanate compound, on a surface of a metal substrate or a plastic substrate.

2. The bioactive implant according to claim 1, wherein the apatite layer is plurally present on the metal substrate or the plastic substrate.

3. A manufacturing method of a bioactive implant, including coating a surface of a metal substrate or a plastic substrate with a coating liquid containing at least crystalline calcium apatite fine particles, an aqueous urethane resin, and a self-emulsifiable isocyanate compound to form an apatite layer.
